(19) United States
(12) Patent Application Publication
(10) Pub. No.: US 2017/0128738 A1
(43) Pub. Date: May 11, 2017

(54) NEURONAL STIMULATOR WITH MICRON RESOLUTION

(71) Applicant: Weinberg Medical Physics, Inc., North Bethesda, MD (US)

(72) Inventor: Irving N. WEINBERG, Bethesda, MD (US)

(21) Appl. No.: 15/343,755

(22) Filed: Nov. 4, 2016

Related U.S. Application Data
(60) Provisional application No. 62/251,859, filed on Nov. 6, 2015.
(60) Provisional application No. 62/253,861, filed on Nov. 11, 2015.

(51) Int. Cl. A61N 2/00; A61B 5/00; A61N 1/36; A61B 18/04; A61B 5/055; A61B 5/05

(52) U.S. Cl. CPC A61N 2/006; A61B 5/055; A61B 5/0515; A61N 1/3605; A61B 18/04; A61B 5/0035; A61B 2018/00577

(57) ABSTRACT

An apparatus and method for stimulating one or more cells within a subject's brain by one or more magnetizable particles under the influence of at least one coil, wherein the coil is located outside the body of the subject, and is used to visualize and move the one or more magnetizable particles and to visualize the subject's underlying anatomy containing the one or more cells.

### CROSS REFERENCE AND PRIORITY CLAIM

This patent application claims priority to U.S. Provisional Applications 62/251,859 filed Nov. 6, 2015 and 62/253,861 filed Nov. 11, 2015, the disclosures of which being incorporated herein by reference in their entirety.

### FIELD OF USE

Disclosed embodiments enable stimulation of neurons in a human or animal brain for research or clinical purposes.

### SUMMARY

Disclosed embodiments provide an apparatus and method for stimulating one or more neurons within a subject's brain by one or more magnetizable particles under the influence of at least one coil.

In accordance with at least one embodiment, the coil is located outside the body of the subject, and is used to visualize and move the one or more magnetizable particles and to visualize the subject's underlying anatomy containing the neurons.

### BRIEF DESCRIPTION OF THE FIGURES

The detailed description particularly refers to the accompanying figures in which:

FIG. 1 illustrates an example of an apparatus for stimulation of cells in a brain in accordance with a disclosed embodiment.

FIG. 2 illustrates an example of a methodology for stimulation of cells in a brain in accordance with a disclosed embodiment.

### DETAILED DESCRIPTION

The past decade has seen many innovations aimed at understanding the structure and connections in the animal and human brain. A major innovation was the field of optogenetics, in which animals were genetically modified so that their neurons were sensitive to light, and in which light sources could be placed near neurons to activate them. Other investigators have noticed that focused ultrasound and direct mechanical stimulation can activate nerves (see, the article by J Lee et al in the March 2015 publication in the journal Experimental Neurobiology entitled “Ca2+ entry is required for mechanical stimulation-induced ATP release from astrocytes;” hereby incorporated by reference in its entirety).

In accordance with at least one embodiment, an apparatus may include and utilize one or more magnetizable particles introduced into the nervous system of a subject. The term “magnetizable” means that the particle includes materials that are, or can be made, magnetic. The introduction into the subject's nervous system may be performed via the cribriform plate or via injection into the spinal canal, or other means. For the purposes of this specification, it should be noted that the word “cribiform” may be alternately spelled as “cribriform”.

It is understood that the term particle encompasses material of a size having its largest dimension being a millimeter in length or smaller, and more preferably, its largest dimension being 100 microns in length or smaller.

It is understood that although the specification primarily discusses the use of magnetizable particles in the brain, the method and apparatus may apply to nervous tissues outside of the central nervous system, or to organs and tissues other than nervous tissues.

In accordance with at least one embodiment, the magnetizable particles may be transported into the nervous system under the control of operations with coils, for example as described in the US Patent Application US 20140309479 entitled “System, Method and Equipment for Implementing Temporary Diamagnetic Propulsive Focusing effect with Transient Applied Magnetic Field Pulses” (incorporated by reference in its entirety) by Weinberg et al, or in disclosures concerning transport of magnetizable particles across barriers, as in US Patent Application US 20130204120, entitled “Equipment and Methodologies for Magnetically-Assisted Delivery of Therapeutic Agents Through Barriers,” (incorporated by reference in its entirety) by Weinberg et al. As an example, magnetizable particles could be introduced into the central nervous system through intranasal administration and across the cribriform plate.

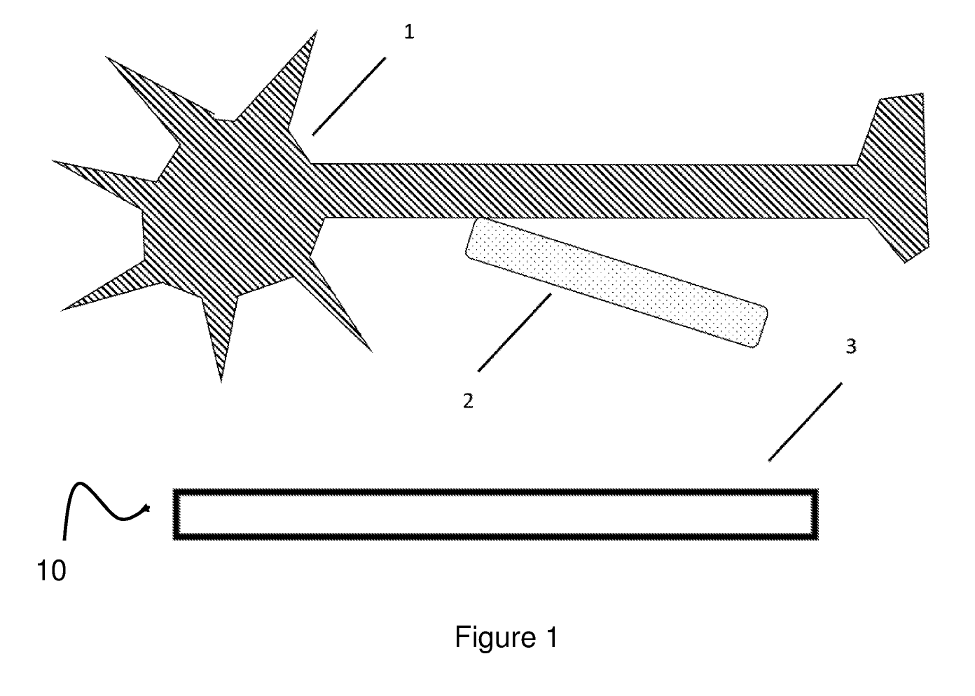

As illustrated in FIG. 1, the apparatus 10 is provided to stimulate one or more neurons 1 using one or more magnetizable particles 2 under the influence of at least one coil 3.

In accordance with at least one embodiment, the at least one coil 3 may be located outside the body of the subject, and may be used to visualize and move the one or more particles 2 and to visualize the subject's underlying anatomy containing one or more neurons 1. For the purpose of this specification, the term “visualize” is intended to convey the process of collecting an image, and the phrase “visualize the location” is intended to convey the process of collecting an image demonstrating the physical location of an object in that image.

The one or more particles 2 may be between 1 and 1000 microns in diameter and between 1 and 1000 microns in length. The magnetizable particles 2 may be manipulated wirelessly by a magnetic field applied by the at least one coil 3 outside of the subject's body. The manipulation may be accomplished through operations described in US patent application US 20140309479 by Weinberg et al, entitled “System, Method and Equipment for Implementing Temporary Diamagnetic Propulsive Focusing Effect with Transient Applied Magnetic Field Pulses” (incorporated herein in its entirety).

The one or more magnetizable particles 2 may be translated through the application of magnetic pulses as in the above referenced and incorporated patent applications.

The at least one magnetizable particle 2 may be visualized, preferably along with underlying anatomy of the subject, through fast magnetic pulse sequences enabled by U.S. Pat. No. 8,154,286, entitled “Apparatus and Method for Decreasing Bio-effects of Magnetic Fields,” to Weinberg and patents and applications related by priority claims (incorporated by reference in their entirety). These pulse sequences may employ MRI or magnetic particle imaging methods.

The one or more magnetizable particles may be vibrated in order to mechanically stimulate at least one neuron through application of an external magnetic field that changes in time. The effect of this stimulation may be measured via MRI techniques (for example reflecting blood oxygenation level dependence) or via optical techniques such as calcium-channel-activated contrast agents, or by observing the behavioral responses of an animal or human. It is understood that sound waves may be generated through motion of the one or more magnetizable particles, and that neuronal stimulation may be as a result of such sound waves. It is understood that the vibration of the particle is effected by changing a magnetic field applied from outside the body. It is understood that heat may be generated through motion of the particle, and that stimulation may be as a result of such heat.

Although the specification mentions stimulation of neurons, it is clear that the same method may be used to stimulate other types of cells (for example, in the gut or endocrine system, where mechanicoreceptors are present). Cells in cancer (i.e., neoplasms) may also be affected by vibration, as is known in the field of high-intensity focused ultrasound, and so the method could be used to modulate cancer growth.

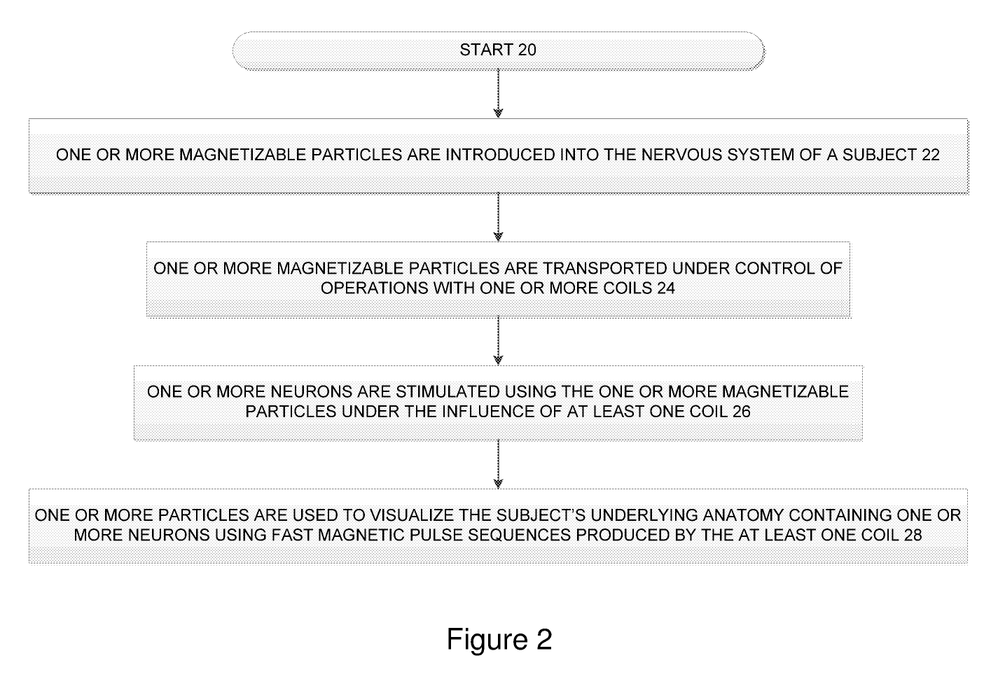

FIG. 2 illustrates an example of a methodology for stimulation of cells in a brain in accordance with a disclosed embodiment. As shown in FIG. 2, the method may begin at 20, at which one or more magnetizable particles are introduced into the nervous system of a subject. Control then proceeds to 22, at which the one or more magnetizable particles are transported under control of operations with one or more coils. Control then proceeds to 24, at which one or more cells are stimulated using the one or more magnetizable particles under the influence of at least one coil. Control then proceeds to 26, at which the one or more particles are used to visualize the subject's underlying anatomy containing one or more cells using fast magnetic pulse sequences produced by the at least one coil. These pulse sequences may employ MRI or magnetic particle imaging methods.

In accordance with at least one embodiment, the one or magnetizable particles may be used to deliver an electrical pulse to its surroundings by application of radiofrequency or other electromagnetic energy from a source (e.g., antenna or coil) external to the body, and which is focused or otherwise transmitted or conveyed by the particle.

In accordance with at least one embodiment, the one or magnetizable particles may contain or be in contact with a small electrical generator in order to deliver an electrical pulse to its surroundings. The generator may be in the form of a glucose fuel cell or a fuel cell operating on another chemical. The generator may be in the form of a tribological material generating electricity upon activation by a magnetic field applied from a source (e.g., coil) external to a body.

In accordance with at least one embodiment, the one or magnetizable particles may stimulate one or more cells, e.g., neurons, through emanation of visible or other forms of light (e.g., infrared or ultraviolet light). The light may be generated through inclusion of a phosphorescent material in or on the particle.

In accordance with at least one embodiment, the one or magnetizable particles may open up physiological barriers (for example, the blood-brain barrier) as a result of motion or vibration of the particle or other energy emitted by the particle under the influence of an applied magnetic field. Once the barrier is open, particles or other substances may pass.

In accordance with at least one embodiment, the one or magnetizable particles may ablate unwanted tissues (for example, cancer) as a result of motion or vibration of the particle or other energy emitted by the particle under the influence of an applied magnetic field. For the purpose of this specification, the term “ablate” is intended to represent causing non-viability of the tissue or the inability of the cells in the tissue to reproduce.

In accordance with at least one embodiment, one or magnetizable particles may stimulate one or more cells, e.g., neurons, through dispersal of chemicals from the interior or surface of the magnetizable particle. The dispersal may be triggered or accelerated by a magnetic field applied from a source (e.g., coil) external to a body.

In accordance with at least one embodiment, a location of the at least one magnetizable particle may be visualized with magnetic resonance imaging or magnetic particle imaging.

In accordance with at least one embodiment, images of the subject's anatomy including the at least one cell may be obtained using magnetic resonance imaging techniques and a location of the at least one magnetizable particle may be superimposed on an image of the subject's anatomy including the at least one cell.

In accordance with at least one embodiment, a location of the at least one magnetizable particle may be varied using a magnetic field applied from outside of the body.

In accordance with at least one embodiment, a response of the stimulation of the at least one cell at the location of another cell, which may be a neuron.

It should be understood that the operations explained herein may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of components of an instrument for applying magnetic fields described herein to manipulate one or more particles may be provided using software instructions that may be stored in a tangible, non-transitory storage device such as a non-transitory computer readable storage device storing instructions which, when executed on one or more programmed processors, carry out he above-described method operations and resulting functionality. In this case, the term non-transitory is intended to preclude transmitted signals and propagating waves, but not storage devices that are erasable or dependent upon power sources to retain information.

Accordingly, such an instrument may include one or more controllable electromagnetic field sources and a controller that enables control of resulting magnetic fields as described herein. In one such implementation, one or more gradient coils may be utilized under the control of a controller to enables control of the gradient to produce one or magnetic fields using at least one coil driver, wherein one or more coils are provided for transmitting RF energy into a tissue sample of a body part as part of diagnostic, prognostic, and/or treatment

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments of the present invention. Such alternative storage devices should be considered equivalents.

While certain illustrative embodiments have been described, it is evident that many alternatives, modifications, permutations and variations will become apparent to those skilled in the art in light of the foregoing description. While illustrated embodiments have been outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

As a result, it will be apparent for those skilled in the art that the illustrative embodiments described are only examples and that various modifications can be made within the scope of the invention as defined in the appended claims.

I claim:

1. An apparatus comprising:
a magnetizable particle introduced into a subject's body in a vicinity of a cell in the body; and
at least one coil positioned external to the body,
wherein the magnetizable particle is moved by a magnetic field created by the at least one coil and affects the cell.

2. A method for affecting at least one cell in a body of a subject by at least one magnetizable particle, the method comprising:
introducing the at least one magnetizable particle into the subject's body;
causing motion of the at least one magnetizable particle; and
causing an effect stimulating the at least one cell by the motion of the at least one magnetizable particle.

3. The method of claim 2, wherein the cell is in the subject's nervous system.

4. The method of claim 2, wherein the cell in in the subject's digestive system.

5. The method of claim 2, wherein the cell in in the subject's endocrine system.

6. The method of claim 2, wherein the cell is neoplastic.

7. The method of claim 2, further comprising visualizing a location of the at least one magnetizable particle with magnetic resonance imaging.

8. The method of claim 2, further comprising visualizing a location of the at least one magnetizable particle with magnetic particle imaging.

9. The method of claim 2, further comprising obtaining images of the subject's anatomy including the region of the at least one cell using magnetic resonance imaging techniques and superimposing a location of the at least one magnetizable particle on an image of the subject's anatomy including the region containing the at least one cell.

10. The method of claim 2, further comprising varying a location of the at least one magnetizable particle using a magnetic field applied from outside of the body.

11. The method of claim 2, wherein stimulation is effected by motion of the at least one magnetizable particle.

12. The method of claim 2, wherein stimulation is effected by electrical energy emanating from the at least one magnetizable particle.

13. The method of claim 2, wherein stimulation is effected by electromagnetic energy emanating from the at least one magnetizable particle.

14. The method of claim 2, wherein the at least one magnetizable particle is introduced into the subject's nervous system through intranasal administration and across the cribriform plate.

15. The method of claim 2, wherein a physiological barrier is opened as a result of motion of the particle or of energy emanating from the at least one magnetizable particle.

16. The method of claim 2, wherein unwanted tissues are ablated as a result of motion of the particle or of energy emanating from the at least one magnetizable particle.

17. An apparatus for stimulating at least one cell in a body of a subject, the apparatus comprising:
at least one magnetizable particle for introduction into the subject's body; and
at least one coil located outside the subject's body that produces magnetic fields applied to the at least one magnetizable particle to cause motion of the at least one magnetizable particle,
wherein the motion of the at least one magnetizable particle stimulates the at least one cell.

18. The apparatus of claim 17, further comprising magnetic resonance imaging equipment for visualizing a location of the at least one magnetizable particle within the subject.

19. The apparatus of claim 17, further comprising magnetic particle imaging equipment for visualizing a location of the at least one magnetizable particle within the subject.

20. The apparatus of claim 17, wherein a location of the at least one magnetizable particle is superimposed on an image of anatomy of the at least one cell obtained with magnetic resonance imaging techniques.

21. The apparatus of claim 17, where a location of the at least one magnetizable particle is varied using a magnetic field applied from outside of the body.

22. The apparatus of claim 17, wherein stimulation is effected by motion of the at least one magnetizable particle.

23. The apparatus of claim 17, wherein stimulation is effected by electrical energy emanating from the at least one magnetizable particle.

24. The apparatus of claim 17, wherein the at least one magnetizable particle emanates electromagnetic energy to stimulate the at least one cell.

25. The apparatus of claim 17, wherein the at least one magnetizable particle is introduced into the subject's nervous system through intranasal administration and across the cribriform plate.

26. The apparatus of claim 17, wherein a physiological barrier is opened as a result of motion of the particle or of energy emanating from the at least one magnetizable particle.

27. The apparatus of claim 17, wherein unwanted tissues are ablated as a result of motion of the particle or of energy emanating from the at least one magnetizable particle.
